(19) United States
(12) Patent Application Publication
(10) Pub. No.: US 2008/0200911 A1
(43) Pub. Date: Aug. 21, 2008

(54) Electrical ablation apparatus, system, and method

(76) Inventor: Gary L. Long, Cincinnati, OH (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/706,591

(22) Filed: Feb. 15, 2007

(51) Int. Cl. A61B 18/18

(52) U.S. Cl. 606/34

(57) ABSTRACT

A surgical instrument, such as an endoscopic or laparoscopic instrument, includes an ablation device. The ablation device includes an elongate relatively flexible member having a proximal end and a distal end. The flexible member includes a working channel. A first electrode extends from the working channel at the distal end of the flexible member and is adapted to be endoscopically located in a first position relative to a tissue treatment region. A second electrode is adapted to be percutaneously located in a second position of the tissue treatment region. The first and second electrodes are adapted to couple to an electrical waveform generator and to receive an electrical waveform sufficient to ablate tissue located between the first and second electrodes.

### CROSS REFERENCE TO RELATED APPLICATION

The present application is related to concurrently-filed U.S. patent application Ser. No. ______, entitled ELECTROPORATION ABLATION APPARATUS, SYSTEM, AND METHOD, by Gary Long, Attorney Docket No. 07034/END6107USNP, which is incorporated herein by reference in its entirety.

### BACKGROUND

Electrical therapy techniques have been employed in medicine to treat pain and other conditions. Electrical ablation techniques have been employed in medicine for removing diseased tissue or abnormal growths from the body. Electrical therapy probes comprising electrodes may be required to electrically treat diseased tissue. The electrodes may be introduced into the patient percutaneously to the tissue treatment region by passing the electrodes through the skin of the patient. If the at least two electrical therapy electrodes are introduced only percutaneously, however, the relative position between these electrodes may be limited. Nevertheless, there is a need for improved medical instruments to electrically ablate or destroy diseased tissue, such as cancer, or abnormal growths from the body. There may be a need for such electrical therapy techniques to be performed endoscopically.

### SUMMARY

In one general aspect, the various embodiments are directed to an ablation device. In one embodiment, the ablation device comprises an elongate relatively flexible member having a proximal end and a distal end. The flexible member comprises a working channel. A first electrode extends from the working channel at the distal end of the flexible member. The first electrode is adapted to be endoscopically located in a first position relative to a tissue treatment region. A second electrode is adapted to be percutaneously located in a second position of the tissue treatment region. The first and second electrodes are adapted to couple to an electrical waveform generator and to receive an electrical waveform sufficient to ablate tissue located between the first and second electrodes.

### FIGURES

The novel features of the various embodiments are set forth with particularity in the appended claims. The various embodiments, however, both as to organization and methods of operation, together with further objects and advantages thereof, may best be understood by reference to the following description, taken in conjunction with the accompanying drawings as

FIG. 1 illustrates one embodiment of an electrical ablation system.

FIG. 2 is an enlarged view of one embodiment of an electrical ablation probe of the electrical ablation system shown in FIG. 1

FIG. 3 illustrates the use of one embodiment of the electrical ablation system to treat diseased tissue on the liver.

FIG. 4 is a sectional view taken along the longitudinal axis of one embodiment of the electrical ablation system shown in FIG. 1.

FIG. 5 is a sectional view taken along line 5-5 of one embodiment of the electrical ablation system shown in FIG. 4.

FIG. 6 is a sectional view taken along line 6-6 of the rotation tube of one embodiment of the electrical ablation system shown in FIG. 4.

FIG. 7 shows a distal portion of one embodiment of the electrical ablation system shown in FIG. 1 inserted into a hollow body or natural opening of a patient.

FIG. 8 is a diagram of one embodiment of a control loop for one embodiment of an electrical (e.g., IRE or RF) therapy procedure to treat diseased tissue as described herein.

FIG. 9 is a graphical representation (graph) of the electric field strength (along the y-axis) as a function of the distance from an electrical therapy electrode under various conductivity environments near the diseased tissue.

FIG. 10 is a close up of the graph shown in FIG. 9.

### DESCRIPTION

The various embodiments described herein are directed to electrical therapy ablation devices. The electrical therapy ablation devices comprise probes and electrodes that can be positioned in a tissue treatment region of a patient either endoscopically or transcutaneously (percutaneously), and in some embodiments a combination thereof. An endoscopic electrode is inserted through a working channel of an endoscope. A transcutaneous or percutaneous electrode has a sharp point to facilitate insertion through the skin of a patient and to enhance local current density at a target site during treatment. The placement and location of the electrodes can be important for effective and efficient therapy. Once positioned, the electrical therapy electrodes deliver electrical current to the treatment region. The electrical current is generated by a control unit or generator external to the patient and typically has particular waveform characteristics, such as frequency, amplitude, and pulse width. Depending on the diagnostic or therapeutic treatment rendered, the probes may comprise one electrode containing both a cathode and an anode or may contain a plurality of electrodes with at least one serving as a cathode and at least one serving as an anode.

Electrical therapy ablation may employ electroporation or electropermeabilization techniques where an externally applied electric field (electric potential) significantly increases the electrical conductivity and permeability of a cell plasma membrane. Electroporation is the generation of a destabilizing electric potential across such biological membranes. In electroporation, pores are formed when the voltage across the cell plasma membrane exceeds its dielectric strength. Electroporation destabilizing electric potentials are generally in the range of several hundred volts across a distance of several millimeters. Below certain magnitude thresholds, the electric potentials may be applied across a biological membrane as a way of introducing some substance into a cell, such as loading it with a molecular probe, a drug that can change the function of the cell, a piece of coding DNA, or increasing the uptake of drugs in cells. If the strength of the applied electrical field and/or duration of exposure to it are suitably chosen, the pores formed by the electrical pulse reseal after a short period of time, during which extracellular compounds have a chance to enter into the cell. Thus, below a certain threshold, the process is reversible and the potential does not permanently damage the cell membrane. This process may be referred to as reversible electroporation (RE).

On the other hand, excessive exposure of live cells to large electric fields can cause apoptosis and/or necrosis—the processes that result in cell death. Thus, the excessive exposure of live cells to large electric fields may be referred to as irreversible electroporation (IRE) because the cells die when exposed to such excessive electrical fields or potentials across the cell membranes.

Electroporation may be performed with devices called electroporators. These appliances create the electric current and send it through the cell. Electroporators may comprise two or more metallic (e.g., aluminum) electrodes connected to an energy source. The energy source generates an electric field having a suitable characteristic waveform output in terms of frequency, amplitude, and pulse width.

Endoscopy means looking inside and refers to looking inside the human body for medical reasons. Endoscopy may be performed using an instrument called an endoscope. Endoscopy is a minimally invasive diagnostic medical procedure used to evaluate the interior surfaces of an organ by inserting a small tube into the body, often, but not necessarily, through a natural body opening. Through the endoscope, an operator may see abnormal or diseased tissue such as lesions and other surface conditions. The endoscope may have a rigid or a flexible tube and in addition to providing an image for visual inspection and photography, the endoscope may be adapted and configured for taking biopsies, retrieving foreign objects, and introducing medical instruments to a tissue treatment region. Endoscopy is a vehicle for minimally invasive surgery.

The embodiments of the electrical therapy ablation devices may be employed for treating diseased tissue, tissue masses, tissue tumors, and lesions (diseased tissue). More particularly, the electrical therapy ablation devices may be employed in minimally invasive therapeutic treatment of diseased tissue. The electrical therapy ablation devices may be employed to deliver energy to the diseased tissue to ablate or destroy tumors, masses, lesions, and other abnormal tissue growths. In one embodiment, the electrical therapy ablation devices and techniques described herein may be employed in the treatment of cancer by quickly creating necrosis and destroying live cancerous tissue in-vivo. Minimally invasive therapeutic procedures to treat diseased tissue by introducing medical instruments to a tissue treatment region through a natural opening of the patient are known as Natural Orifice Translumenal Endoscopic Surgery (NOTES)™.

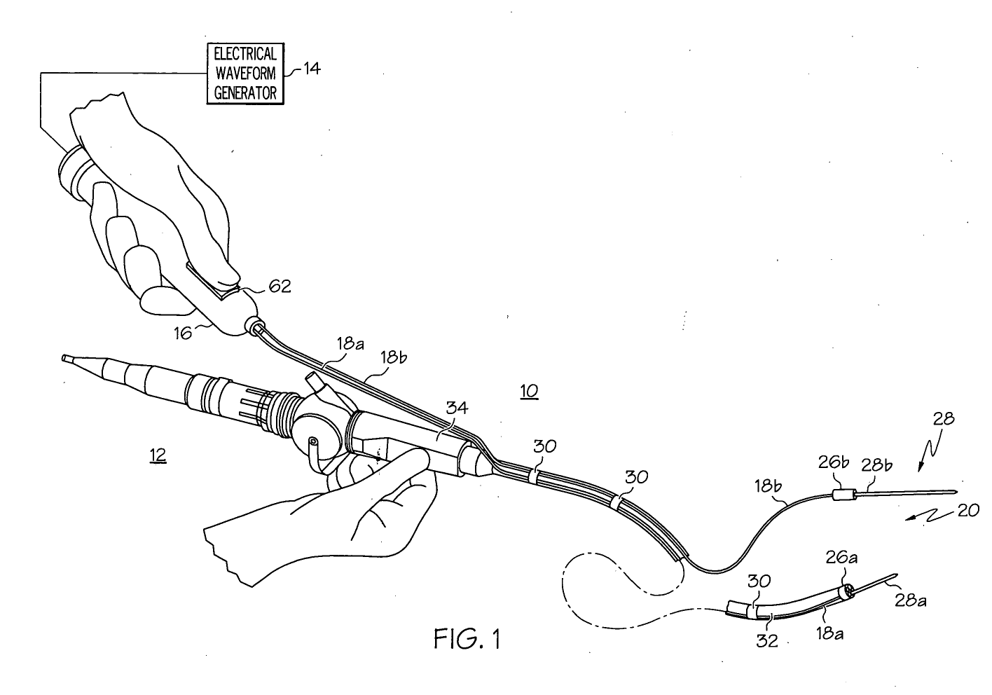

FIG. 1 illustrates one embodiment of an electrical ablation system 10. The electrical ablation system 10 may be employed to electrically treat diseased tissue such as tumors and lesions inside a patient. The electrical ablation system 10 may be configured to be positioned within a natural opening of the patient such as the colon or the esophagus and can be passed through the natural opening to reach a tissue treatment region. The illustrated embodiment of the electrical ablation system 10 may be used to treat diseased tissue via the colon or the esophagus of the patient, for example. The tissue treatment region may be located in the esophagus, colon, liver, breast, brain, and lung, among others. The electrical ablation system 10 can be configured to treat a number of lesions and ostepathologies comprising metastatic lesions, tumors, fractures, infected site, inflamed sites, and the like. Once positioned at the target tissue treatment region, the electrical ablation system 10 can be configured to treat and ablate diseased tissue in that region. In one embodiment, the electrical ablation system 10 may be adapted to treat diseased tissue, such as cancers, of the gastrointestinal (GI) tract or esophagus that may be accessed orally. In another embodiment, the electrical ablation system 10 may be adapted to treat diseased tissue, such as cancers, of the liver or other organs that may be accessible trans-anally through the colon and/or the abdomen.

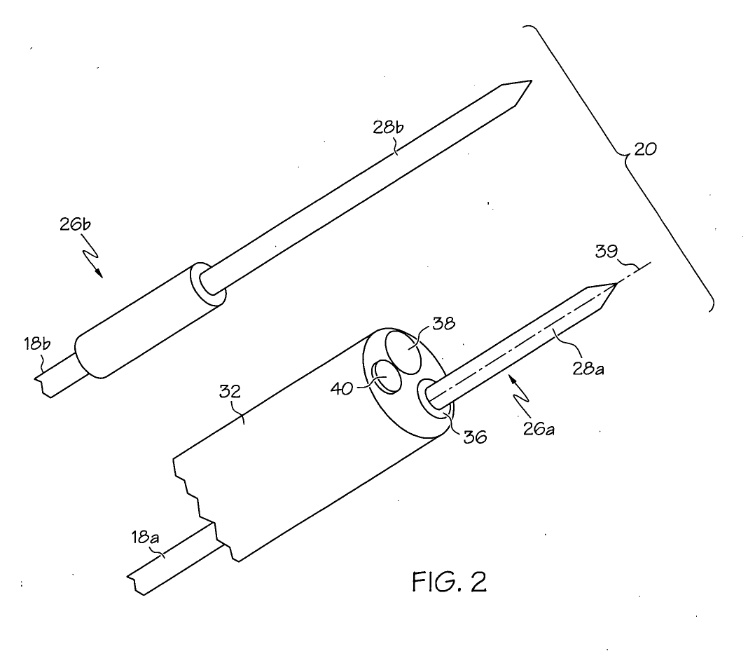

One embodiment of the electrical ablation system 10 may be mounted on a flexible endoscope 12 (also referred to as endoscope 12), such as the GIF-100 model available from Olympus Corporation. The flexible endoscope 12 includes an endoscope handle 34 and an elongate relatively flexible shaft 32. The electrical ablation system 10 generally comprises an electrical ablation probe 20, a plurality of electrical conductors 18, a handpiece 16 having a switch 62, and an electrical waveform generator 14. The electrical ablation probe 20 is located at a distal end of the flexible shaft 32 and the electrical conductors 18 may attach to the flexible shaft 32 using a plurality of clips 30. The electrical ablation probe 20 comprises electrical therapy probes 26a,b to deliver electrical energy to a desired tissue treatment region. The electrical therapy probes 26a,b comprise one or more electrical therapy electrodes 28a,b. A first electrical therapy probe 26a comprises a first electrical therapy electrode 28a and is electrically connected to a first electrical conductor 18a. The first electrical therapy electrode 28a extends through a bore in the flexible shaft 32 such as a working channel 36 (FIG. 2) of the endoscope 12. The first electrical therapy electrode 28a is introduced to the desired tissue treatment region endoscopically. The first electrical therapy probe 26a may be referred to herein as an endoscopic electrical therapy probe. A second electrical therapy probe 26b comprises a second electrical therapy electrode 28b and is electrically connected to a second electrical conductor 18b. The second electrical therapy electrode 28b is introduced to the desired tissue treatment region transcutaneously by piercing the skin covering the tissue treatment region. The second electrical therapy probe 26b may be referred to herein as a transcutaneous electrical therapy probe. Once the first and second electrical therapy electrodes 28a,b are located at respective first and second positions in the tissue treatment region, manual operation of the switch 62 of the handpiece 16 electrically connects or disconnects the electrical therapy electrodes 28a,b to the electrical waveform generator 14. Alternatively, the switch 62 may be mounted on, for example, a foot switch (not shown).

In one embodiment, the electrical waveform generator 14 may be a conventional, bipolar/monopolar electrosurgical IRE generator such as one of many models commercially available, including Model Number ECM 830, available from BTX Molecular Delivery Systems Boston, Mass. The IRE generator generates electrical waveforms having predetermined frequency, amplitude, and pulse width. The application of these electrical waveforms to the cell membranes of the diseased tissue causes the diseased cells to die. Thus, the IRE electrical waveforms may be applied to the cell membranes of diseased tissue in the tissue treatment region in order to kill the diseased cells and ablate the diseased tissue. IRE electrical waveforms suitable to destroy the cells of diseased tissues are generally in the form of direct current (DC) electrical pulses delivered at a frequency in the range of 1-20 Hz, amplitude in the range of 100-1000 VDC, and pulse width in the range of 0.01-100 ms. For example, an electrical waveform having amplitude of 500 VDC and pulse duration of 20 ms may be delivered at a pulse repetition rate or frequency of 10 HZ to destroy a reasonably large volume of diseased tissue. Unlike RF ablation systems which require high powers and energy input into the tissue to heat and destroy, IRE requires very little energy input into the tissue, rather the destruction of the tissue is caused by high electric fields. It has been determined that in order to destroy living tissue, the electrical waveforms have to generate an electric field of at least 30,000 V/m in the tissue treatment region. The embodiments, however, are not limited in this context.

In one embodiment, the electrical waveform generator 14 may comprise a radio frequency (RF) waveform generator. The RF generator may be a conventional, bipolar/monopolar electrosurgical generator such as one of many models commercially available, including Model Number ICC 350, available from Erbe, GmbH. Either a bipolar mode or monopolar mode may be used. When using the bipolar mode with two electrodes, one electrode is electrically connected to one bipolar polarity, and the other electrode is electrically connected to the opposite bipolar polarity. If more than two electrodes are used, the polarity of the electrodes may be alternated so that any two adjacent electrodes have opposite polarities. Either the bipolar mode or the monopolar mode may be used with the illustrated embodiment of the electrical ablation system 10. When using the bipolar mode with two electrical therapy electrodes 28a,b the first electrode 28a may be electrically connected to one bipolar polarity, and the second electrode 28b may be electrically connected to the opposite bipolar polarity (or vice-versa). If more than two electrical therapy electrodes 28 are used, the polarity of the electrodes 28 is alternated so that any two adjacent electrodes have opposite polarities.

In either case, the electrical (e.g., the IRE or RF) waveform generator 14, when using the monopolar mode with two or more electrical therapy electrodes 28, a grounding pad is not needed on the patient. Because a generator will typically be constructed to operate upon sensing connection of ground pad to the patient when in monopolar mode, it can be useful to provide an impedance circuit to simulate the connection of a ground pad to the patient. Accordingly, when the electrical ablation system 10 is used in monopolar mode without a grounding pad, an impedance circuit can be assembled by one skilled in the art, and electrically connected in series with one of the electrical therapy electrodes 28a,b that would otherwise be used with a grounding pad attached to a patient during monopolar electrosurgery. Use of an impedance circuit allows use of the IRE generator in monopolar mode without use of a grounding pad attached to the patient.

FIG. 2 is an enlarged view of one embodiment of an electrical ablation probe 20 of the electrical ablation system 10 shown in FIG. 1. The first electrical therapy electrode 28a is introduced to the tissue treatment region endoscopically and extends through the distal end of the flexible shaft 32. In one embodiment, the first electrode 28a protrudes from the distal end of an internal lumen extending between the proximal and distal ends of the flexible endoscope 12. In one embodiment, the internal lumen may be the working channel 36 of the endoscope 12. The first electrode 28a may be rotatable about a central axis 39 within the working channel 36 to facilitate locating the electrode 28a in a first position in the tissue treatment region. The second electrical therapy electrode 28b is introduced percutaneously to the target tissue treatment region. The second electrode 28b is located in a second position in the tissue treatment region. Introducing the second electrode 28b percutaneously allows the first and second electrodes 28a,b to be spaced further apart than if the two electrodes 28a,b were both introduced endoscopically. Spacing the first and second electrodes 28a,b further apart allows the electrodes to surround a larger diseased tissue region and generate an electric field over a much larger tissue treatment region. In this manner, the operator can surround the entire tissue treatment region of a cancerous lesion, a polyp, or a tumor, for example. Introducing at least one of the electrodes (e.g., the first electrode 28a) endoscopically enables the operator to accurately locate the target diseased tissue region using endoscopic visualization feedback by employing at least one light source 40 and a viewing port 38 located on the distal end of the endoscope 12. The electrodes 28a,b may be energized with the electrical waveform generator 14 to deliver an IRE or an RF electrical waveform to treat the diseased tissue located between the first and second electrodes 28a,b. Because the electrodes 28a,b are located in the tissue treatment region independently, the operator has much more flexibility in positioning the electrodes 28a,b relative to the tissue treatment region.

The electrical conductors 18a,b are electrically insulated from each other and surrounding structures, except for the electrical connections the respective electrical therapy electrodes 28a,b. The distal end of the flexible shaft 32 of the flexible endoscope 12 may comprise the light source 40, the viewing port 38, and the working channel 36, where the first electrode 28a may be passed therethrough. The viewing port 38 transmits an image within its field of view to an optical device such as a charge coupled device (CCD) camera within the flexible endoscope 12 so that an operator may view the image on a display monitor (not shown). In the embodiment shown in FIG. 2, the distal end of flexible shaft 32 is proximal to the first electrode 28a within the field of view of the flexible endoscope 12 thus enabling the operator to see the tissue treatment region to be treated near the first electrode 28a. This technique provides a more accurate way to locate the first electrode 28a in the tissue treatment region.

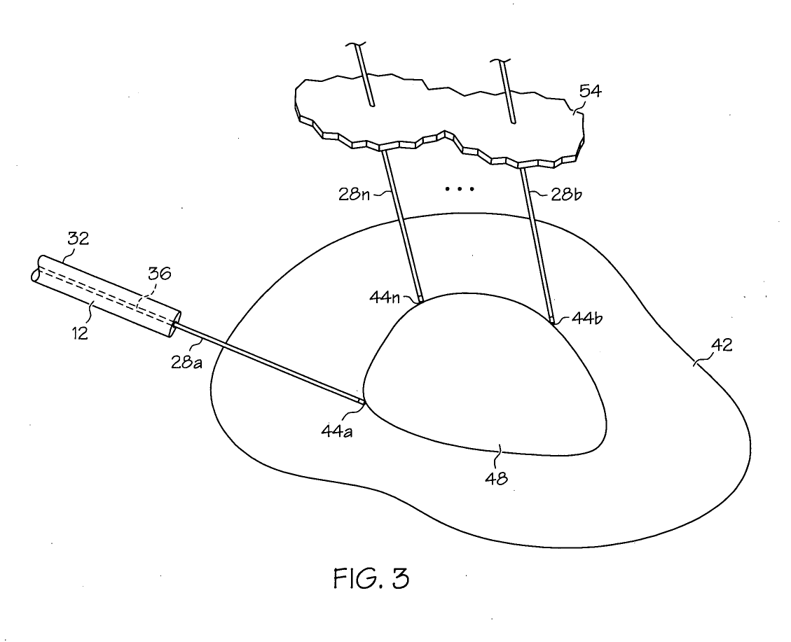

FIG. 3 illustrates the use of one embodiment of the electrical ablation system 10 to treat a diseased tissue 48 on the liver 42. In the embodiment illustrated in FIG. 3, the flexible shaft 32 of the endoscope 12 has been introduced to the tissue treatment region trans-anally into the abdomen. The first electrical therapy electrode 28a is introduced through the working channel 36 of the flexible shaft 32. The operator positions the first electrical therapy electrode 28a using endoscopic visualization so that the diseased tissue 48 to be treated lies within the field of view of the flexible endoscope 12. The operator locates the first electrode 28a located in a first position 44a at a perimeter edge of the diseased tissue 48. The operator then positions or introduces the second electrode 28b percutaneously through the skin 54 of the patient such that the second electrode 28b is located at a second position 44b at a perimeter edge of the diseased tissue 48. Once the first and second electrodes 28a,b are located at the desired first and second positions 44a,b, the operator may energize the electrodes 28a,b with the electrical waveform generator 14 to deliver an IRE or an RF waveform suitable to destroy the diseased tissue 48. For example, in an IRE embodiment, the first and second electrodes 28a,b may be energized with an electrical waveform having amplitude of approximately 500 VDC and a pulse width of approximately 20 ms at a frequency of approximately 10 Hz. In this manner, the diseased tissue 48 may be destroyed. This procedure may be efficient and repeated to destroy relatively larger portions of the diseased tissue. Those skilled in the art will appreciate that similar techniques may be employed to treat any other diseased tissues accessed trans-anally through the colon and/or the abdomen and/or accessed orally through the esophagus or the stomach. Therefor, the embodiments are not limited in this context.

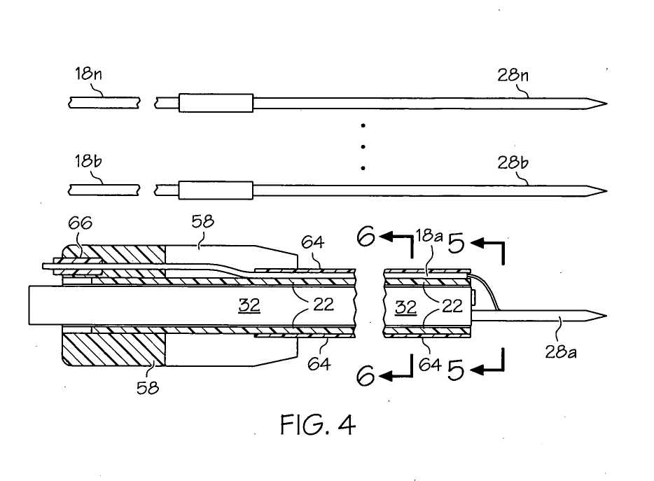

FIG. 4 is a sectional view taken along the longitudinal axis of one embodiment of the electrical ablation system 10 shown in FIG. 1. The distal portion of the flexible shaft 32 is located inside a rotation tube 22 of the electrical ablation system 10. The first electrical conductor 18a passes through a strain relief 66 of a rotation knob 58. In the illustrated embodiment an external tube 64 or sheath may be located over the flexible shaft 32 such that the first electrical conductor 18a passes between the external tube 64 and the rotation tube 22. The first electrical conductor 18a connects electrically to the first electrical therapy electrode 28a. The rotation tube 22 rotatably joins the rotation knob 58. The operator can rotatably orient the first electrode 28a, even after insertion into the natural opening (e.g., trans-anally into the abdomen) by remotely rotating the probe 26a. The electrical ablation probe 20 is located within the field of view of the flexible endoscope 12 to enable the operator to see on a display monitor the tissue that is located between the electrodes 28.

With reference to FIGS. 3 and 4, in one embodiment, multiple electrical therapy probes 26a-n each connected to respective electrical conductors 18a-n and electrical therapy electrodes 28a-n may be employed to surround the diseased tissue region around multiple points 44a-n. This may be beneficial in destroying relatively larger portions of the diseased tissue. Once the electrodes 28a-n are located at the desired positions 44a-n, the operator may energize the electrodes 28a-n with the electrical waveform generator 14 to deliver an IRE or an RF waveform suitable to destroy the diseased tissue 48. For example, in an IRE embodiment, the electrodes 28a-n may be energized with an electrical waveform having amplitude of approximately 500 VDC and a pulse width of approximately 20 ms at a frequency of approximately 10 Hz. In this manner, the diseased tissue 48 may be destroyed.

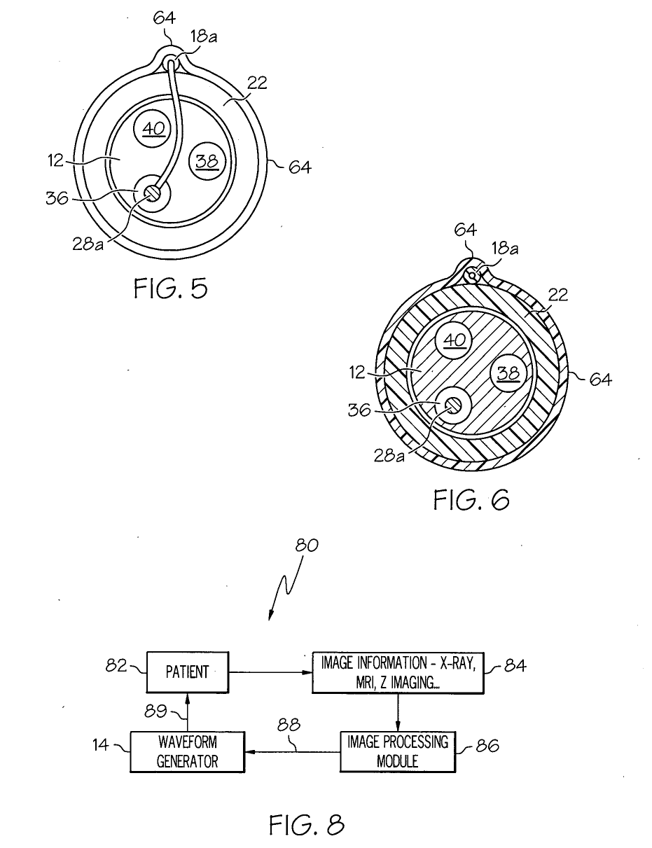

FIG. 5 is a sectional view taken along line 5-5 of one embodiment of the electrical system 10 shown in FIG. 4. The electrical conductors 18a,b connect to the respective electrical therapy electrodes 28a,b. The rotation tube 22 retains the flexible shaft 32. The inside diameter of the rotation tube 22 is larger than the outer diameter of the flexible endoscope 12 to allow rotation of the rotation tube 22 while holding the flexible endoscope 12 stationary, or vice versa. The first electrode 28a extends outwardly from the distal end of the flexible shaft 32 through the working channel 36. The second electrode 28b is connected to the waveform generator 14 through the electrical conductor 18b and is provided outside of the flexible endoscope 12 to be introduced to the tissue treatment region percutaneously. In this embodiment, the operator may endoscopically view the tissue between the electrodes 28a,b as illuminated by the light source 40 and viewed through the viewing port 38.

FIG. 6 is a sectional view taken along line 6-6 of the rotation tube 22 of one embodiment of the electrical ablation system 10 shown in FIG. 4. The external tube 64 or sheath and the rotation tube 22 assemble and retain the first electrical conductor 18a as already described. The light source 40, the viewing port 38, and the working channel 36 of the flexible endoscope 12 are shown.

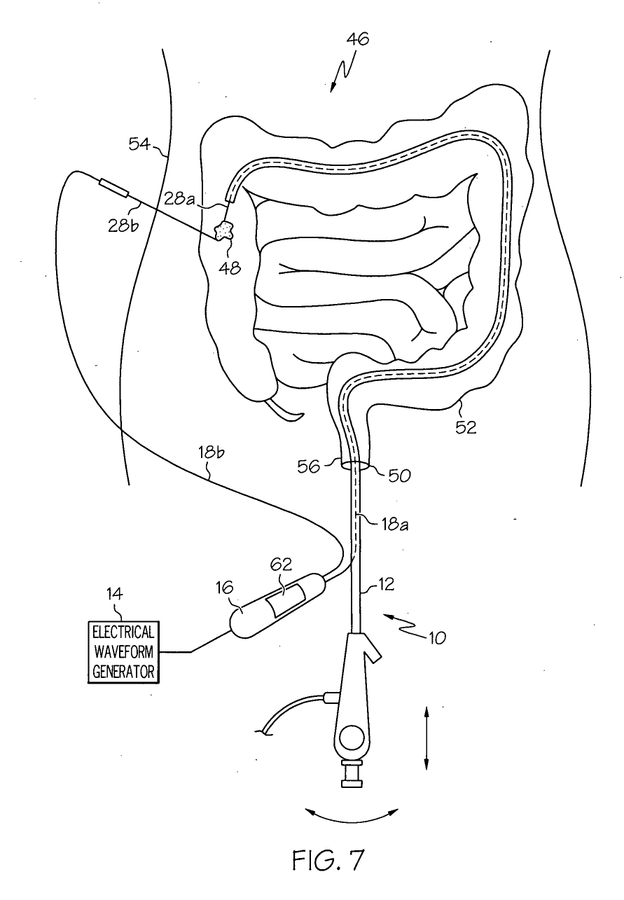

FIG. 7 shows a distal portion of one embodiment of the electrical ablation system 10 shown in FIG. 1 inserted into a hollow body or natural opening of a patient. The electrical ablation system 10 is inserted into the colon 46 through the anus 50. The colon 46 includes a sphincter muscle 52 disposed between the anus 50 and the rectum 56. The electrical ablation system 10 is maneuvered through several turns through the colon 46. The electrical ablation system 10 is introduced to the diseased tissue 48 through the colon 46.

The operator may treat the diseased tissue 48 using the embodiment of the electrical ablation system 10 comprising the electrical ablation probe 20 with the electrical therapy electrode 28a introduced endoscopically and the electrical therapy electrode 28b introduced transcutaneously or percutaneously through the skin 54 as previously discussed with reference to FIGS. 1-7 as follows. The operator inserts the flexible shaft 32 of the endoscope 12 into the anus 50 and maneuvers it through the colon 46. The operator uses endoscopic visualization through the viewing port 38 to position the first electrical therapy electrode 28a next to the diseased tissue 48 on the liver 42 to be treated. If the diseased tissue 48 is on the liver 42, the distal end of the endoscope 12 can be advanced into the sigmoid colon. Once in the sigmoid colon an instrument such as a needle knife can be advanced through the lumen of the endoscope. The needle knife can then cut an opening through the sigmoid colon and into the peritoneal space (under visualization). The endoscope can then be advance into the peritoneal space and manipulate until the liver is in view. The operator then introduces the second electrical therapy electrode 28b transcutaneously though the skin 54 to the diseased tissue 48. This can be done under visualization using the view from the endoscope or with fluoroscopy. The transcutaneous electrode is then advanced into the liver. The first and second electrodes 28a,b are placed in intimate contact with the diseased tissue 48 to be treated within the field of view of the flexible endoscope 12. While watching through the viewing port 38, the operator actuates the switch 62, electrically connecting the electrodes 28a,b to the waveform generator 14 through the conductors 18a,b. Electric current then passes through the portion of the diseased tissue 48 positioned between the electrodes 28a,b and within the field of view. When the operator observes that the tissue in the field of view has been ablated sufficiently, the operator deactuates the switch 62 to stop the ablation. The operator may reposition either the endoscopic electrode 28a or the transcutaneous electrode 28b for subsequent tissue treatment, or may withdraw the electrical ablation probe 20 (together with the flexible endoscope 12).

FIG. 8 is a diagram of one embodiment of a control loop 80 for one embodiment of an electrical (e.g., IRE or RF) therapy procedure to treat diseased tissue as described herein. As previously discussed, the electrical therapy procedure may be effective in quickly creating necrosis of live tissue and destroying diseased (e.g., cancerous) tissue in-vivo. Real time information feedback about the size in volume of a necrotic zone may be helpful during an electrical therapy procedure for focal treatment of diseased tissue 48.

Prior to an electrical therapy procedure, a patient 82 will have an image of the diseased tissue 48 taken for clinical purposes in an effort to reveal, diagnose, or examine the diseased tissue 48 and to identify its location more precisely. The image information 84 will generally include geometric information about the volume of the diseased tissue 48. The image information 84 is provided to an image processing module 86 to calculate the volume of the diseased tissue 48 and to display a virtual model of the diseased tissue 48 on a monitor. The image processing module 86 may comprise, for example, image processing software applications such as Comsol Multiphysics available by Comsol, Inc. to receive the image information 84, extract the geometric information, and determine (e.g., calculate) the voltage required to treat the proper volume and outline of the necrotic zone required to treat the diseased tissue 48. The image processing module 86 creates a virtual model of a treatment zone necessary to treat the diseased tissue 48. The image processing module 86 then determines waveform parameters 88 of a suitable electrical waveform necessary to destroy the diseased tissue 48. The waveform parameters 88 include the frequency, amplitude, and pulse width of the electrical waveform to be generated by the waveform generator 14. The waveform generator 14 would then generate the suitable electrical waveform to destroy the diseased tissue 48 based on the calculated electrical waveform parameters 88.

The image processing module 86 also comprises image processing software applications such as Matlab available by MathWorks, Inc. to receive the image information 84 and the virtual model and display an image of the diseased tissue 48 overlaid with an image of the virtual model. The overlaid images enable the operator to determine whether the calculated electrical waveform parameters 88 are suitable for destroying the diseased tissue 48, whether too strong or too weak. Thus, the electrical waveform parameters 88 may be adjusted such that the virtual model image substantially over-lays the entire diseased tissue image. The calculated 88 parameters are provided to the waveform generator 14 and the diseased tissue may be treated with an electrical waveform 89 based on the calculated parameters 88 as discussed herein. After the diseased tissue 48 is treated with the electrical waveform 89, a new image of the diseased tissue 48 can be generated to determine the extent or effectiveness of the treatment. The cycle may be repeated as necessary to ablate the diseased tissue 48 as much as possible.

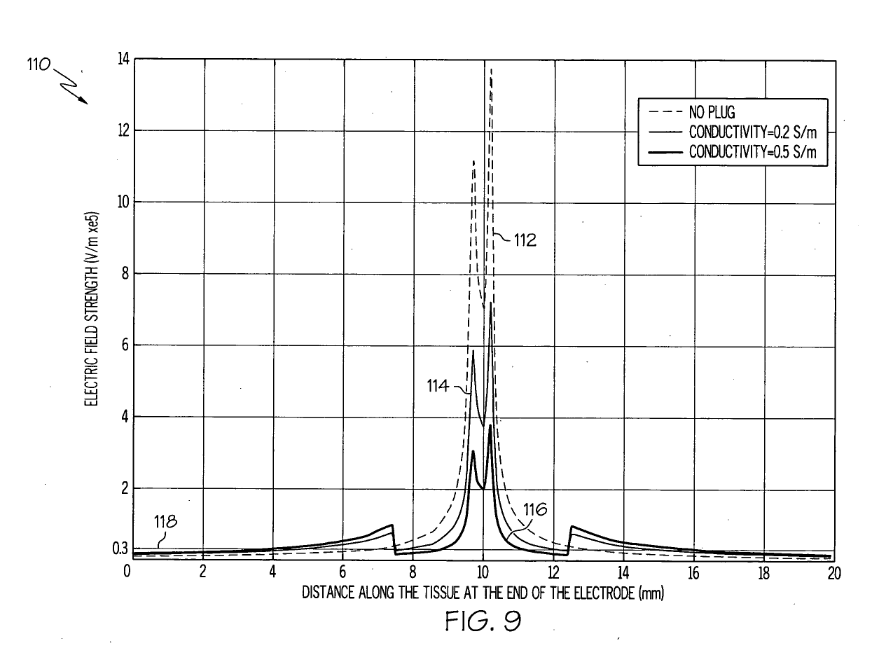

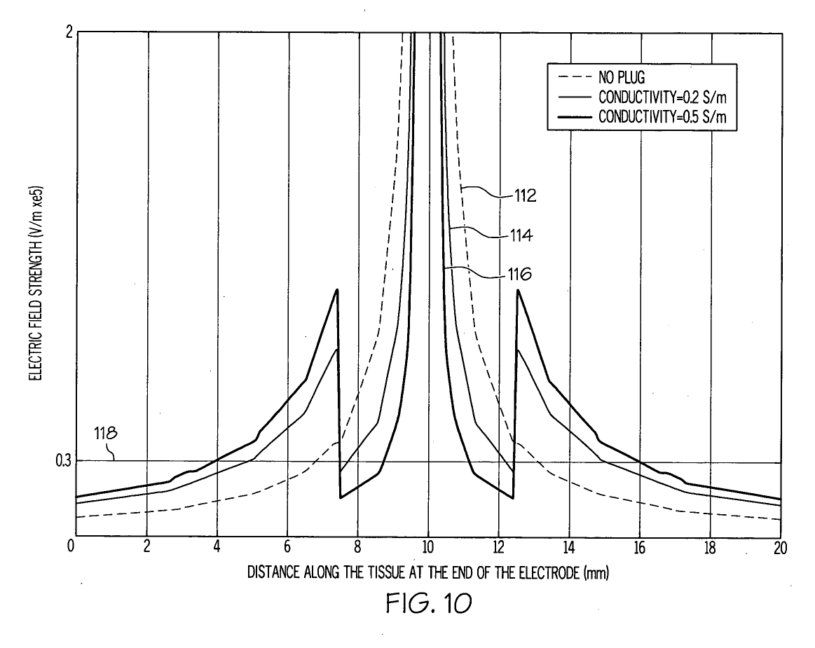

FIG. 9 is a graphical representation 110 (graph) of the electric field strength (along the y-axis) as a function of the distance from an electrical therapy electrode 28a,b under various conductivity environments near the diseased tissue 48. FIG. 10 is a close up of the graph 110 shown in FIG. 9. In electrical therapy of diseased tissue 48, the volume of tissue that can be destroyed by an electrical waveform (e.g., the necrotic zone) may be defined by a minimum electric field strength applied to the tissue treatment region. The electric field strength in the tissue treatment region varies throughout the tissue as a function of the applied electrical waveform parameters such as frequency, amplitude, and pulse width as well as the conductivity of the tissue in the treatment region. When a single electrical therapy electrode 28a or 28b is located in a first position in the tissue treatment region of interest and a return pad is placed at a distance relatively far from the first position, an electric field is generated around the respective electrode 28a or 28b when it is energized with a particular electrical waveform. The magnitude of the electric field, however, diminishes rapidly in the radial direction away from the electrode 28a,b. When the electrodes 28a,b are placed relatively close together, a larger pattern of tissue can be destroyed. Injecting a fluid having a higher conductivity than the tissue into the tissue treatment region extends the electric field of sufficient strength to destroy the tissue radially outwardly from the electrode 28a,b. Thus, the addition of a fluid having higher conductivity than the tissue to be treated creates a larger tissue destruction zone (e.g., necrotic zone) by extending the electric field radially outwardly from the electrodes 28a,b.

The graph 110 illustrates the electric field strength, along the y-axis, as a function of the radial distance from the electrical therapy electrode 28a,b. The y-axis is labeled in units of volts/meter (V/m×e⁵) and the x-axis is labeled in units of mm. The graph 110 illustrates a family of three functions with conductivity as a parameter. A first function 112 illustrates the electric field strength as a function of the radial distance from one of the electrodes 28a,b with no conductivity plug introduced into the tissue treatment region. A second function 114 illustrates the electric field strength as a function of the radial distance from one of the electrodes 28a,b with a conductivity plug of 0.2 S/m introduced in the tissue treatment region. A third function 116 illustrates the electric field strength as a function of the radial distance from one of the electrodes 28a,b with a conductivity plug of 0.5 S/m introduced in the tissue treatment region. As shown in the graph 110, the peak electric field strength of each of the functions 112, 114, 116 decreases with increased conductivity in the tissue treatment region in proximity to the electrode 28a,b. However, the threshold 118 of each of the functions 112, 114, 116 where the electric field strength drops below the minimum threshold 118 of electric field strength required for tissue destruction becomes wider as the conductivity increases. In other words, increasing the conductivity of the tissue in the tissue treatment region extends the range of an effective electric field to destroy tissue or creates a larger necrotic zone. In one embodiment, the minimum electric field strength threshold 118 is approximately 30,000 V/m.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments of the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although the various embodiments of the invention have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An ablation device comprising:
an elongate relatively flexible member having a proximal end and a distal end, the flexible member comprising a working channel;
a first electrode extending from the working channel at the distal end of the flexible member, the first electrode is adapted to be endoscopically located in a first position relative to a tissue treatment region; and
a second electrode adapted to be percutaneously located in a second position of the tissue treatment region;
wherein the first and second electrodes are adapted to couple to an electrical waveform generator and to receive an electrical waveform sufficient to ablate tissue located between the first and second electrodes.

2. The ablation device of claim 1, wherein the first and second electrodes are adapted to receive an irreversible electroporation (IRE) waveform from an IRE generator.

3. The ablation device of claim 1, wherein the first and second electrodes are adapted to receive a radio frequency (RF) waveform from an RF generator.

4. The ablation device of claim 1, comprising:
at least a third electrode adapted to be percutaneously located in a third position of the tissue treatment region, wherein the third electrode is adapted to couple to the electrical waveform generator and to receive an electrical waveform sufficient to ablate tissue located between the first, the second, and the at least third electrodes.

5. The ablation device of claim 1, comprising:
at least one illuminator supported on the device and positioned to illuminate tissue; and
an image sensor supported on the device and positioned to image tissue therethrough.

6. A method comprising:
receiving image information of a diseased tissue region in a patient;
determining a volume and outline of a necrotic zone required to treat the diseased tissue based on the image information; and
determining waveform parameters to be generated by an electrical waveform generator suitable to destroy the diseased tissue.

7. The method of claim 6, comprising:
extracting geometric information from the image information; and
determining the volume and outline of the necrotic zone required to treat the diseased tissue based on the geometric information.

8. The method of claim 6, comprising:
providing the waveform parameters to an electrical waveform generator.

9. The method of claim 6, comprising:
determining amplitude, frequency, and pulse width waveform parameters suitable to destroy the diseased tissue.

10. An ablation system, comprising:
an ablation device comprising:
an elongate relatively flexible member having a proximal end and a distal end, the flexible member comprising a working channel;
a first electrode extending from the working channel at the distal end of the flexible member, the first electrode is adapted to be endoscopically located in a first position relative to a tissue treatment region; and
a second electrode adapted to be percutaneously located in a second position of the tissue treatment region; and
an electrical waveform generator electrically coupled to the ablation device;
wherein the first and second electrodes are adapted to receive an electrical waveform sufficient to ablate tissue located between the first and second electrodes.

11. The ablation system of claim 10, wherein the electrical waveform generator comprises an irreversible electroporation waveform generator.

12. The ablation system of claim 10, wherein the electrical waveform generator comprises a radio frequency generator.

13. The ablation system of claim 10, wherein the ablation device comprises at least a third electrode adapted to be percutaneously located in a third position of the tissue treatment region, wherein the third electrode is adapted to couple to the electrical waveform generator and to receive an electrical waveform sufficient to ablate tissue located between the first, the second, and the at least third electrodes.

14. The ablation system of claim 10, wherein the ablation device comprises at least one illuminator supported on the device and positioned to illuminate tissue; and an image sensor supported on the device and positioned to image tissue therethrough.

15. The system of claim 10, wherein the electrical waveform generator is to receive electrical waveform parameters from an image processing module; the electrical waveform parameters are suitable to destroy the diseased tissue; and the electrical waveform parameters are determined based image information of the diseased tissue region in a patient.

16. The system of claim 15, wherein the electrical waveform parameters are determined based on a volume and outline of a necrotic zone required to treat the diseased tissue based on the image information; and the waveform parameters are to be generated by the electrical waveform generator.

17. The system of claim 16, wherein the volume and outline of the necrotic zone are determined from geometric information extracted from the image information.

18. The system of claim 15, wherein the electrical waveform parameters comprise amplitude, frequency, and pulse width of an electrical waveform suitable to destroy the diseased tissue.

19. A method comprising:
introducing an elongate relatively flexible member having a proximal end and a distal end into a natural opening of a patient, the flexible member comprising a working channel and a first electrode extending from the working channel at the distal end of the flexible member, the first electrode is adapted to be endoscopically located in a first position relative to a tissue treatment region;
introducing a second electrode percutaneously in a second position of the tissue treatment region; and
ablating the tissue located between the first and second electrodes.

20. The method of claim 19, comprising:
ablating the tissue with an irreversible electroporation (IRE) waveform from an IRE generator electrically coupled to the first and second electrodes.

21. The method of claim 19, comprising:
introducing at least a third electrode percutaneously in a third position of the tissue treatment region, wherein the third electrode is adapted to couple to the electrical waveform generator and to receive an electrical waveform sufficient to ablate tissue located between the first, the second, and the at least third electrodes.

22. The method of claim 19, comprising:
obtaining image information of the tissue treatment region in the patient;
determining a volume and outline of a necrotic zone required to treat the tissue in based on the image information; and
determining waveform parameters to be generated by an electrical waveform generator suitable to destroy the diseased tissue.

23. The method of claim 22, comprising:
extracting geometric information from the image information; and
determining the volume and outline of the necrotic zone required to treat the diseased tissue based on the geometric information.

24. The method of claim 23, comprising:
providing the waveform parameters to an electrical waveform generator.

25. The method of claim 23, comprising:
determining amplitude, frequency, and pulse width waveform parameters suitable to destroy the diseased tissue.

26. The method of claim 19, comprising:
ablating the tissue device with a radio frequency (RF) waveform from an RF generator electrically coupled to the first and second electrodes.

27. The method of claim 19, comprising introducing a conductive liquid into the tissue treatment region, wherein the conductive liquid has a conductivity that is relatively higher than the conductivity of the tissue in the tissue treatment region.

28. A method comprising:
obtaining a surgical instrument, wherein the surgical instrument comprises:
an elongate relatively flexible member having a proximal end and a distal end, the flexible member comprising a working channel;
a first electrode extending from the working channel at the distal end of the flexible member, the first electrode is adapted to be endoscopically located in a first position relative to a tissue treatment region; and
a second electrode adapted to be percutaneously located in a second position of the tissue treatment region;
wherein the first and second electrodes are adapted to couple to an electrical waveform generator and to receive an electrical waveform sufficient to ablate tissue located between the first and second electrodes;
sterilizing the surgical instrument; and
storing the surgical instrument in a sterile container.
